(19) United States
(12) Patent Application Publication
(10) Pub. No.: US 2004/0018554 A1
(43) Pub. Date: Jan. 29, 2004

(54) Methods and compositions for detection of bovine spongiform encephalopathy and variant creutzfeldt-jacob disease

(76) Inventor: Larry R. Green, Tacoma, WA (US)

Correspondence Address: Richard A. Nakashima, BLAKELY, SOKOLOFF, TAYLOR & ZAFMAN LLP, 7th Floor, 12400 Wilshire Boulevard, Los Angeles, CA, 90025, US

(21) Appl. No.: 10/128,608

(22) Filed: Apr. 22, 2002

Related U.S. Application Data
(60) Provisional application No. 60/291,477, filed on May 15, 2001.

(51) Int. Cl. G01N 33/53

(52) U.S. Cl. 435/7.1

(57) ABSTRACT

The present invention discloses compositions and methods for the detection of infective agents (prions) associated with transmissible spongiform encephalopathies. More particularly, the present invention involves compositions and methods for detection and diagnosis of “mad cow” disease and vCJD. In certain embodiments, prions are treated to remove bound lipids before immunodetection. In other embodiments, hydrophobic probes are used to collect prions from oral or anal tissue. Preferred embodiments of the invention involve the use of arrays of binding moieties, such as antibodies, with varying degrees of affinity and specificity for the infective agent. The presence of prions in biological samples may be determined by the pattern of binding of infective agent to the array. The prions may be distinguished from other proteins of similar or identical amino acid sequence, but different secondary, tertiary or quaternary structure, by the different patterns of binding to the array.

### CROSS REFERENCE TO RELATED APPLICATIONS

The present application claims the benefit under 35 U.S.C. §119(e) of provisional patent application No. 60/291,477, filed on May 15, 2001.

### BACKGROUND OF THE INVENTION

1. Field of the Invention

The present invention relates generally to compositions and methods for detecting transmissible spongiform encephalopathies (TSEs) in mammals, including humans and non-human mammals. More particularly, the present invention relates to detecting bovine spongiform encephalopathy (“mad cow disease” or BSE), variant Creutzfeldt-Jakob disease (vCJD) and other prion associated diseases using immunoassays that can detect the presence of prions in a sample.

2. Description of Related Art

The transmissible spongiform encephalopathies (TSE) constitute a group of neurodegenerative diseases. Human TSEs include Creutzfeldt-Jakob disease (CJD), Gerstmann-Straussler-Scheinker syndrome, Fatal Familial Insomnia (FFI), and kuru (Prusiner, 1998). TSEs known to occur in non-human mammals include sheep scrapie, bovine spongiform encephalopathy, transmissible mink encephalopathy, and chronic wasting disease of captive deer and elk (Prusiner, 1998). Transmissible spongiform encephalopathies are characterized by spongiform degeneration, astrocyte gliosis and transmission when inoculated into laboratory animals including primates, rodents, and transgenic mice. Although the histopathologic effects of TSEs are relatively similar, the clinical symptoms can vary. Thus, CJD typically presents as a progressive dementia, while scrapie and BSE generally manifest as ataxias (Prusiner, 1998).

Recently, a variety of early onset CJD, referred to as “vCJD,” has been linked to consumption of meat from cows infected with “mad cow” disease (Horwich and Weissman, 1997). As there is presently no known cure for this disease, efforts at disease prevention have focused on identification and destruction of infected animals and restrictions on export of infected animals and animal products. These measures have resulted in substantial economic disruption in affected countries.

Controlling the spread of vCJD and mad cow disease has been hampered by the lack of a sensitive and specific test for the presence of the infective agent (also known as prions), and the length of time required to obtain results from tested animals. Presently available tests typically rely on histopathologic analysis of brain tissue from animals that exhibit clinical symptoms of the disease. Although more recent diagnostic tests purportedly work with samples of cerebral-spinal fluid (CSF), obtaining such samples is painful, difficult and not well suited for large scale screening efforts. Because present tests do not detect the presence of the infective agent early in disease progression, discovery of an infected animal results in destruction of entire herds on the affected farm and surrounding areas. Undoubtedly many non-infected animals are destroyed along with the disease carriers.

There is a substantial need in the field for a diagnostic test for the presence of the infective agent responsible for vCJD and mad cow disease. Such a test should preferably be capable of detecting prions in samples from clinically asymptomatic animals or humans, with results obtainable within hours or at most a few days from sample collection. Even more preferably, such a test should be capable of detecting prions in biological samples that can be obtained by relatively non-invasive means, such as blood, saliva, urine, feces, adipose tissue punch or swabs from oral or rectal tissues.

### SUMMARY OF THE INVENTION

The present invention addresses deficiencies in the art by providing compositions and methods for the detection of the infective agent causing vCJD and mad cow disease. In certain embodiments, the compositions and methods involve one or more binding moieties, such as antibodies, that bind with high affinity and selectivity to the infective agent. In preferred embodiments, the antibodies of use in the practice of the present invention are monoclonal, although the use of polyclonal antibodies is contemplated within the scope of the present invention.

Other embodiments involve the use of sample pretreatment to increase the sensitivity and specificity of prion detection. In preferred embodiments, such pretreatments may involve the use of detergents or other agents to remove bound lipids from the prion protein. The change in secondary and tertiary structure involved in formation of the infectious form of prion proteins increases the hydrophobicity of the protein, as reflected by its decreased solubility in aqueous solutions. (Horwich and Weissman, 1997; Borman, 1998). As hydrophobic proteins, prions may preferentially localize as integral membrane proteins within lipid bilayers, while free prion proteins in solution may preferentially exist as micellar aggregates with bound lipid. Because lipid binding can screen antigenic epitopes from exposure to the surrounding medium, lipid removal would increase the availability of such epitopes for antibody binding, resulting in an increased sensitivity and specificity of immunodetection techniques.

In the most preferred embodiments, non-denaturing detergents are used to remove bound lipids without changing the secondary or tertiary structure of the prion protein. Such non-denaturing detergents are well known in the art. Non-limiting examples include Triton X-100 and other Triton detergents, NP-40, Brij, Tween, octyl-β-thio-glucopyranoside, CHAPS, CHAPSQ, sodium cholate and other cholate type detergents and Genapol X-80. Other non-limiting examples of non-denaturing compounds that can be used for lipid removal include Non Detergent Sulfobetaines (NDSB). In certain embodiments, lipid may be removed by treatment with an alcohol, formaldehyde or other fixative before antibody binding. The invention is not limited to use of the listed detergents or non-detergent agents. Any treatment that is effective to remove bound lipids from prion proteins may be used within the scope of the instant invention.

In alternative embodiments, lipids can be removed by enzymatic treatment, such as with lipases, phospholipases, sphingolipases and other such enzymes known in the art. The skilled artisan will realize that delipidation may reduce the solubility of prion proteins in aqueous solution. In order to enhance the aqueous solubility of delipidated prion protein, it may be necessary to keep low concentrations of a non-denaturing detergent or other amphipathic molecule in the solution. In certain embodiments, it may be desirable to use a protein carrier such as plasminogen, apolipoprotein or serum albumin to increase solubility of prion proteins in aqueous solution.

The hydrophobic nature of the prion protein may be advantageously used for collection of samples for analysis. It has been reported that the infectious form of prion proteins are preferentially bound to plasminogen (Fischer et al., 2000), presumably through hydrophobic interactions. It is proposed that the hydrophobic partitioning of prions may be used to enhance sample collection, for example by swabbing tissues that may contain high levels of prions. Non-limiting examples of such collection methods include using a swab or other probe coated with a hydrophobic layer, such as a detergent, lipid, a hydrophobic synthetic polymer or a hydrophobic protein such as plasminogen or apolipoprotein. Within the scope of the present invention, any hydrophobic substance that is effective to selectively collect prion proteins from a tissue surface may be used. In preferred embodiments, the hydrophobic-coated probe is used to swab oral tissues, such as the tongue, throat or tonsils, or anal tissues. It is further expected that prion proteins may preferentially localize in tissues with a high fat content, such as adipose tissue. In other embodiments, an adipose tissue punch may be used to collect samples enriched in prion proteins.

In certain preferred embodiments, prions may be detected by a pattern of binding to multiple binding moieties that exhibit differing degrees of affinity and/or specificity for prions. Such patterns of binding may be particularly useful where different strains or different species-specific prions may be present in a sample. The specific detection of prions compared to other cross-reactive species such as non-infective proteins of the same amino acid sequence but different secondary and tertiary structure may be facilitated by pattern recognition upon binding to an array of binding moieties. Although individual binding agents (such as antibodies) may show some degree of cross-reactivity, the pattern of binding to the array should be definitive for infectious prions compared with similar non-infectious proteins. Although preferred embodiments involve multiple distinct binding moieties, it is contemplated within the scope of the present invention that single binding moieties, such as a single antibody, may also be used. Arrays comprising multiple copies of a single binding moiety are also contemplated within the scope of the invention.

Other embodiments of the present invention concern identifying prions and distinguishing them from other cross-reactive molecules or aggregates. In preferred embodiments, identification occurs using a data analysis system, such as a pattern recognition system. In such embodiments, it is anticipated that the prions may bind to more than one binding moiety. Other analytes present in biological samples, such as non-infectious forms of the infective agent (PrP-C), may bind to one or more binding moieties. Prions in a sample may be distinguished from non-specific binding or from binding of cross-reactive moieties by the pattern of optical signals detected from a test section array. Utilizing arrays comprising different antibodies of differing specificities and affinities for prions, samples containing prions will exhibit unique patterns of optical signals that may be identified by pattern recognition software or other data analysis means known in the art. In the most preferred embodiments, the data analysis means comprises custom software specifically designed to identify and distinguish prions from other components of biological samples.

In some embodiments, the disclosed methods concern the utilization of detection methods and apparatus designed for use with a multiple binding moiety format (e.g., an array). Non-limiting examples of such methods and apparatus are well known in the art (e.g., U.S. Pat. Nos. 5,827,748; 6,192,168; 6,197,599; 6,258,606; 6,294,392; 6,365,418). In preferred embodiments, the methods and apparatus of use in detection are as disclosed in U.S. patent application Ser. No. 09/974,089, filed Oct. 10, 2001, the entire text of which is incorporated herein by reference.

U.S. patent application Ser. No. 09/974,089 discloses a controlled flow, portable biosensor apparatus, comprising a fluidics cube attached to a waveguide. The surface of the waveguide may be attached to multiple binding moieties, each of which can be selective or specific for a different analyte and/or a different epitope of the same analyte. Binding moieties may be arranged on the waveguide surface in specific patterns of spots and each spot may be individually detected for analysis of analyte binding. In certain embodiments, an excitatory light beam, such as a diode laser, may be used to excite luminescent tags attached to a binding moiety and/or analyte. Emitted light may be detected by a detector, which may be operably coupled to a computer for information processing and data storage and transmission. In preferred embodiments, the detectors of use are capable of detecting optical signals, such as spectrometers, monochromators, CCD devices, CCD cameras, photomultiplier tubes, photodetector cells, photodiodes, avalanche photodiodes, phototransistors, vacuum photodiodes, silicon photodiodes or even CMOS chips. The skilled artisan will realize that the method of detection is not limiting, but may encompass any method or apparatus known in the art.

Although the preferred binding moieties comprise one or more antibodies, the skilled artisan will realize that the present invention is not limited to use of antibodies for the binding moiety. Any molecule or aggregate that binds with sufficiently high affinity and specificity to the infective agent may be used in the practice of the present invention. Non-limiting examples of such alternative binding moieties include non-infectious forms of the infective agent (PrP-C), chaperones, plasminogen and peptide libraries (e.g., U.S. Pat. Nos. 5,565,332, 5,596,079, 6,031,071 and 6,068,829, incorporated herein by reference).

Other embodiments of the present invention concern compositions comprising antibodies capable of binding to infective agents. Such antibodies may be prepared against discrete structural domains of the infective agent by means well known in the art.

In preferred embodiments, the compositions and methods of the present invention are of use in detecting the presence of prions in biological samples from human or non-human mammals in the early stages of TSE disease progression. In the most preferred embodiments, the compositions and methods may be used to detect the presence of prions in asymptomatic human or non-human mammals. In other preferred embodiments, the non-human mammal is a cow or a sheep. In additional preferred embodiments, the biological samples to be analyzed may be collected by relatively non-invasive means, such as blood, saliva or urine samples, lymph node aspirates or needle biopsy samples, adipose tissue punches, or swabs of oral or rectal tissues, although the analysis of samples such as cerebrospinal fluid or brain tissue are also within the scope of the present invention. Preferably, the process of exposing a sample to binding moiety, detecting binding and analysis of signals is capable of being fully automated, allowing a rapid throughput of samples.

Other embodiments of the present invention concern compositions and methods for therapeutic treatment of TSEs. These involve production of high affinity binding moieties that can bind to and stabilize the non-infectious form of the infective agent. In preferred embodiments, the binding moieties are capable of binding to the infectious form of the infective agent and converting it to a non-infectious form. Alternatively, the binding moiety may prevent binding of PrP-C to PrP-Sc by blocking the binding site for heterodimer formation. Administration of pharmaceutical compositions comprising such high affinity binding moieties is of use for treatment of TSEs.

### BRIEF DESCRIPTION OF THE DRAWINGS

No drawings are necessary for the understanding of the subject matter of the present invention.

### DETAILED DESCRIPTION OF THE PREFERRED EMBODIMENTS

As used herein, the terms “a” and “an” mean one or more than one of an item.

As used herein, the terms “infective agent,” “infectious form of the infective agent,” “prion,” “prion protein” and “PrP-Sc” are used synonymously. The terms generally refer to a form of protein that is associated with the development of transmissible spongiform encephalopathies. The terms may refer to a purified, partially purified, or highly purified form of the protein.

As used herein, the terms “PrP-C” and “non-infectious form of the infective agent” are used synonymously. The terms generally refer to a normal cellular counterpart of prion protein, of similar or identical amino acid sequence but different secondary and tertiary structure. The terms may refer to a purified, partially purified, or highly purified form of the protein.

The term “PrP,” without modification, is used herein to refer to either or both PrP-Sc and PrP-C.

The terms “detection,” “detecting,” “diagnosis” and “diagnostic” are used herein to refer to an assay or procedure that is indicative of the presence of PrP-Sc or predicts the onset of a transmissible spongiform encephalopathy, such as vCJD or mad cow disease. It will be appreciated by those of skill in the art that all assays exhibit a certain level of false positives and false negatives. Even where a positive result in an assay is not invariably associated with the ultimate onset of the disease, the result is of use as it indicates the need for more careful monitoring of the individual and the institution of appropriate containment procedures, reducing risk of infection and transmission through the population. An assay is diagnostic of a transmissible spongiform encephalopathy when the assay results show a statistically significant association or correlation with the ultimate manifestation of symptoms of a transmissible spongiform encephalopathy (e.g., vCJD or mad cow disease). Within the scope of the present invention, any positive assay results for the presence of PrP-Sc in a sample from an individual is presumed to be diagnostic for vCJD or mad cow disease in the individual.

A “biological sample” is a sample containing biological material. The sample may be a fluid or tissue sample from an animal or human, including without limitation a sample of blood, lymphatic fluid, urine, cerebrospinal fluid, feces, a biopsy sample, punch sample, thin section sample, or a sample from any organ, tissue or cell. A “biological sample” may also be a sample of a non-biological material, including without limitation dirt, water, sewage, food or beverage, which is suspected of containing a biological material, such as a virus, bacterium, feces, prion, etc.

“Individual” as used herein refers to a single human or non-human mammal.

The term “array” is used in accordance with its plain and ordinary meaning of an arrangement containing two or more of an item. In preferred embodiments, the array comprises an arrangement of two or more of an item (such as monoclonal antibodies) wherein each item is in a fixed, known location on the array.

As used herein, a “binding moiety” is a molecule or aggregate that has binding affinity for some analyte, such as a prion.

“Binding” refers to an interaction between a target analyte and a binding moiety, resulting in a sufficiently stable complex so as to permit detection of the analyte:binding moiety complex. In certain embodiments, binding may also refer to an interaction between a second molecule and a target analyte. For example, in a sandwich ELISA type of detection assay, the binding moiety is an antibody with affinity for an analyte. After binding of analyte to binding moiety, a second molecule, typically a labeled antibody with an affinity for a different epitope of the analyte, is added and the tertiary complex of first antibody:analyte:second labeled antibody is detected. In alternative embodiments, the first binding moiety may have affinity for a target analyte while the second binding moiety has affinity for the first binding moiety. Although detection may involve the use of a second binding moiety with affinity for an analyte, in alternative embodiments the binary complex of binding moiety with analyte may be directly detected. The skilled artisan will be familiar with a variety of techniques by which an analyte:binding moiety complex may be detected, any of which may be utilized within the scope of the present invention.

As used herein, an “analyte” is a compound, molecule or aggregate of interest, to be detected in a sample. The term “analyte” is used broadly to encompass anything from a portion of a molecule, a single intact molecule, an aggregate of molecules or a complex assembly, such as a virus, cell or bacterium.

Prions

Work by Prusiner identified the infective agent in TSEs as an aberrant protein, known as a “prion” (for proteinaceous infectious particle) (Prusiner, 1998). TSEs are characterized by deposition of prion proteins (designated as PrP-Scrapie or PrP-Sc), the infectious form of the proteins, in the central nervous system of affected individuals. A normal cellular counterpart of PrP-Sc (known as PrP-Cellular or PrP-C), is ubiquitously distributed in the tissues of normal individuals, including brain tissue, as a cell surface glycoprotein of unknown function (Horwich and Weissman, 1997). Although mutant forms of PrP proteins that predispose to TSEs are known, in the majority of cases the infective PrP-Sc apparently has the same amino acid sequence as the non-infective PrP-C (Horwich and Weissman, 1997; Prusiner, 1998).

The most widely accepted model for conversion of the non-infective PrP-C to infective PrP-Sc involves a change in the secondary and tertiary structure of the protein, in the apparent absence of covalent modification. PrP-C is primarily alpha helical in structure, with about 40% alpha helical content and little or no beta sheet (Horwich and Weissman, 1997). The infective PrP-Sc contains 50% beta sheet structure and only about 20% alpha helix (Horwich and Weissman, 1997). Since levels of PrP mRNAs are apparently similar in normal and TSE infected tissues, it is believed that the disease state does not involve activation of PrP gene expression. Rather, the PrP-Sc protein is apparently able to convert normal cellular PrP-C to the infective PrP-Sc conformation, presumably by binding to PrP-C and either catalyzing a transition to or stabilizing the PrP-Sc conformation. The PrP-Sc conformation is reported to be highly resistant to proteases and to other treatments that would be expected to abolish or inhibit protein activity.

The characteristics of PrP-Sc present substantial difficulties for accurate and sensitive detection of PrP-Sc in biological samples. Since the primary structures of PrP-Sc and PrP-C are identical, many binding moieties that show an affinity for PrP-Sc may also exhibit some affinity for PrP-C. Certain embodiments of the present invention address this problem by providing an array of binding moieties that differ in their affinities and specificities for PrP-Sc. By examining the pattern of binding to specific binding moieties, it is possible to separate specific binding to PrP-Sc from non-specific binding to PrP-C or other analytes. Specific detection of PrP-Sc is also facilitated by controlling conditions to avoid denaturation of PrP-C, which would result in the exposure of additional antigenic domains to the binding moiety. The skilled artisan is well aware of denaturing conditions to avoid, such as extremes of temperature, pH, or salt concentration, the presence of denaturing detergents or chaotrophic agents, exposure to ionizing radiation, denaturing chemicals, etc.

The species specificity of PrP-Sc infectivity has also complicated analysis. For example, although the mouse and hamster forms of PrP differ only by 16 out of 254 residues, mice are normally resistant to the widely used 263K strain of hamster prion (Horwich and Weissman, 1997). Mice can be made susceptible to hamster PrP-Sc by transgenic insertion of the hamster gene, either with or without simultaneous disruption of the mouse gene (Horwich and Weissman, 1997). Until the recent observation of vCJD, it was thought that higher primates were resistant to bovine forms of PrP-Sc. This suggests that minor changes in primary structure of PrP may have substantial effects on the ability of PrP-Sc to bind to PrP-C, presumably reflecting differences in the secondary, tertiary or quaternary structures of the two forms of the protein. Since vCJD is caused by bovine PrP-Sc, in preferred embodiments the binding moieties used would be capable of binding to and detecting either human or bovine PrP-Sc. In alternative embodiments, different sets of binding moieties are used to detect bovine PrP-Sc and human PrP-Sc.

Prions are also known to occur in the form of distinct “strains,” that are capable of producing different patterns of incubation time, CNS localization and patterns of proteolytic cleavage of PrP-Sc (Horwich and Weissman, 1997). These strain specific properties appear to reflect conformational differences between the PrP-Sc proteins within a species, resulting in alternative forms of tertiary or quaternary structure, although covalent modification of the different forms has not been ruled out (Horwich and Weissman, 1997). The existence of mutant forms of PrP protein that differ in primary structure also complicates detection and analysis. In preferred embodiments of the present invention, the binding moieties used are capable of binding to and detecting PrP-Sc independent of strain or point mutation. However, it is contemplated within the scope of the present invention that different sets of binding moieties may be used to detect different strains or mutant forms of PrP-Sc. Alternatively, using an array of binding moieties with different affinities and specificities would allow identification of different PrP-Sc strains or mutant forms, by differences in the pattern of optical signals produced upon exposure of the array to a sample.

The reported characteristics of prions, including their apparent high resistance to inactivation by proteases, ionizing radiation and chemicals such as formaldehyde are difficult to reconcile with the identification of prions as proteins. The great majority of proteins are easily inactivated by protease treatment, ionizing radiation and chemical denaturants. Prions are also unusual as an infectious agent, in that the disease can take ten years or longer to develop following infection, with apparently little increase in the amount of prion protein present in infected animals until late in disease progression. These anomalous properties of prions may be explained if the prion protein is present in the host in a cryptic form—one that is resistant to denaturing treatments and to detection by standard immunoassays.

It is proposed that the anomalous properties of prions may be explained by the hydrophobic character of PrP-Sc. During the refolding process, the alpha-helical structure of PrP-C is converted to a primarily beta sheet conformation of PrP-Sc. The PrP-Sc conformation is much more hydrophobic than PrP-C, as evidenced by its lack of solubility in aqueous solution and by its preferential binding to plasminogen, another hydrophobic protein. It is proposed that the PrP-Sc conformation is sufficiently hydrophobic that it normally exists as an integral membrane protein that is buried within lipid bilayers of cells, with little exposure to the aqueous surroundings.

A potential route for uptake of prions, following ingestion of contaminated food, is by adsorption into fatty tissues lining the gastrointestinal tract, such as the tonsils. The hydrophobic nature of the protein would facilitate uptake by phagocytic cells of the immune system, such as polymorphonuclear leukocytes. The bilayer embedded protein would enter the lymphatic chyle and eventually move through the reticuloendothelial system to the blood, with concentration in the spleen. As cells containing embedded prions die and lyse, the prion protein would be localized in lipid micelles, which could fuse with new host cells. Within the micelle environment, the prion protein could also be protected from exposure to the aqueous environment. Eventually, prions would encounter myelinated peripheral nerves, which have a very high lipid content. A cell-to-cell transfer up the nerve to the central nervous system could result. Once in the brain, prions could easily spread through the lipid rich environment. As prions move through this uptake route, they would encounter and bind to naturally occurring PrP-C, converting it to PrP-Sc and thus forming additional prions.

This model predicts that cryptic prion proteins are present in gradually increasing concentrations throughout disease progression. The reason that PrP-Sc is difficult to detect early in progression, or in blood samples in general, is that the protein is buried within lipid bilayers or micelles and so protected from antibody binding. This would also explain the resistance of prions to protease inactivation. A consequence of this is that prions should be detectable in blood or in tissues with a high fat content, like adipose tissue, upon removal of bound lipids. It should also be possible to concentrate prions by adsorption to a hydrophobic probe surface.

The relative insensitivity to protease activity of lipid encapsulated PrP-Sc, compared to PrP-C, has been used to distinguish between the two conformational forms, by detecting protease resistant PrP binding (e.g., U.S. Pat. No. 6,214,565). The present invention may utilize protease treatment of a sample, followed by protease inactivation, before exposing the sample to a binding moiety. Alternatively, the sample may be exposed to an array of binding moieties, the array washed to remove unbound analytes, the binding pattern detected by optical signals, the array treated with protease under conditions that are effective to remove PrP-C but not PrP-Sc, and the binding pattern detected again. In this subtractive process, PrP-Sc may be identified either by the binding pattern after protease treatment, or by the difference in binding pattern before and after protease treatment. Such a subtractive process would be conducted with binding moieties (for example, antibodies) that have been protected from protease activity in the solution. The use of protease treatment of samples to assist in detection of infective agent is contemplated within the scope of the present invention.

The ability to perform rapid, automated analysis of PrP-Sc in biological samples obtained by relatively non-invasive means provides considerable and unexpected advantages for the compositions and methods of the claimed invention over alternative methods for detection and diagnosis of TSEs (e.g., U.S. Pat. Nos. 5,998,149; 6,008,435; 6,033,858; 6,165,784; 6,214,565).

Antibody Production

The present invention provides for the use of PrP-Sc proteins or peptides as antigens for the immunization of animals relating to the production of antibodies. It is envisioned that PrP-Sc proteins, or portions thereof, may be coupled, bonded, bound, conjugated, or chemically-linked to one or more agents via linkers, polylinkers, or derivatized amino acids. This may be performed such that a bispecific or multivalent composition or vaccine is produced. Methods of preparation of such compositions, suitable for administration to human and/or non-human mammals (i.e., pharmaceutically acceptable) are known in the art. Preferred agents for use as carriers are keyhole limpet hemocyanin (KLH) or bovine serum albumin (BSA).

In preferred embodiments, the present invention contemplates one or more antibodies that are immunoreactive with a PrP-Sc molecule, or any portion thereof. An antibody can be a polyclonal or a monoclonal antibody. In a preferred embodiment, an antibody is a monoclonal antibody. Means for preparing and characterizing antibodies are well known in the art (see, e.g., Harlow and Lane, 1988; incorporated herein by reference). In even more preferred embodiments, the PrP-Sc protein is treated to remove bound lipid before attempting to induce antibody formation, thus increasing the antigenicity of PrP-Sc and exposing additional epitopic domains to antibody production.

Polyclonal antibodies are prepared by immunizing an animal with an immunogen comprising PrP-Sc and collecting antisera from that immunized animal. A wide range of animal species can be used for the production of antisera. Typical animals used for production of anti-antisera include, for example, rabbits, mice, rats, hamsters, pigs or horses. Because of the relatively large blood volume of rabbits, a rabbit is a preferred choice for production of polyclonal antibodies, while mice are preferred for monoclonal antibody production.

Antibodies, both polyclonal and monoclonal, specific for isoforms of PrP may be prepared using conventional immunization techniques, as will be generally known to those of skill in the art. A composition containing antigenic epitopes of PrP-Sc can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the antibodies of the present invention will find useful application in standard immunochemical procedures, such as ELISA and Western blot methods and in immunohistochemical procedures such as tissue staining, as well as in other procedures which may utilize antibodies specific to PrP-Sc antigenic epitopes.

The antibodies of the present invention are also useful for the isolation of PrP polypeptides by immunoprecipitation. Immunoprecipitation involves the separation of the target antigen component from a complex mixture, and is used to discriminate or isolate minute amounts of protein. For the isolation of membrane proteins, cells must be solubilized into detergent micelles. Nonionic salts are preferred, since other agents such as bile salts, precipitate at acid pH or in the presence of bivalent cations.

As is well known in the art, a given composition may vary in its immunogenicity. It is often necessary, therefore, to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). Polyclonal antibody production may be monitored by sampling the blood of immunized animals at various times after immunization. Later, booster injections also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified composition comprising PrP-Sc. The immunizing composition is administered in a manner effective to stimulate antibody-producing cells. Cells from rodents such as mice and rats are preferred, however, the use of rabbit, sheep or frog cells is also possible. The use of rats may provide certain advantages (Goding, 1986), but mice are preferred, with the BALB/c mouse being most preferred.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 41, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus (Kohler and Milstein, 1975; 1976), and those using polyethylene glycol (PEG), such as 37% (v/v) PEG, have been described by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two wk. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three wk) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines also could be cultured in vitro, where the mAbs are naturally secreted into the culture medium from which they can be readily obtained in high concentrations. Monoclonal antibodies produced by either means may be further purified, if desired, using filtration, centrifugation, and various chromatographic methods such as HPLC or affinity chromatography.

Although the methods and compositions disclosed herein allow for the production of novel antibodies against PrP-Sc, it is contemplated within the scope of the invention that previously characterized anti-PrP-Sc antibodies (see, e.g., U.S. Pat. Nos. 6,165,784 and 6,214,565) or commercially available antibodies (e.g., Prionics AF, Zurich, Switzerland) may be used to bind to and detect PrP-Sc protein.

Immunodetection of PrP-Sc

Antibodies of the present invention can be used in characterizing the PrP-Sc content of biological samples through techniques such as ELISA, Western blotting or any other immunodetection methods known in the art. This may provide a screen for the diagnosis of TSEs, such as vCJD or BSE, in individual humans or non-human mammals. As discussed above, treatment of samples to remove bound lipid from PrP-Sc is preferred to increase the sensitivity and specificity of immunodetection methods.

The use of the antibodies of the present invention in an ELISA assay is specifically contemplated. In an exemplary embodiment, anti-PrP-Sc antibodies are immobilized on a selected surface, preferably a surface exhibiting a protein affinity such as the wells of a polystyrene microtiter plate, although attachment to other surfaces such as glass slides or cover slips are contemplated within the scope of the invention. After washing to remove incompletely adsorbed material, it is desirable to bind or coat the surface with a non-specific protein that is known to be antigenically neutral with regard to the test antisera, such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the surface, coating with a non-reactive material to reduce background, and washing to remove unbound material, the immobilizing surface is contacted with the sample to be tested in a manner conducive to immune complex (antigen/antibody) formation. Following formation of specific immunocomplexes between the test sample and the bound antibody, and subsequent washing, the occurrence and even amount of immunocomplex formation may be determined by subjecting the same to a second antibody having specificity for PrP-Sc that differs from that of the first antibody. Appropriate conditions preferably include diluting the sample with diluents such as BSA, bovine gamma globulin (BGG), and phosphate buffered saline (PBS)/Tween®. These added agents also tend to assist in the reduction of nonspecific background. The layered antisera is then allowed to incubate for from about 2 to about 4 h, at temperatures preferably on the order of about 25° to about 27° C. Following incubation, the antisera-contacted surface is washed so as to remove non-immunocomplexed material. A preferred washing procedure includes washing with a solution such as PBS/Tween® or borate buffer.

To provide for detection of bound PrP-Sc, the second antibody may have an associated enzyme that will generate a color development upon incubating with an appropriate chromogenic substrate. Thus, for example, one will desire to contact and incubate the second antibody-bound surface with a urease or peroxidase-conjugated anti-IgG for a period of time and under conditions which favor the development of immunocomplex formation (e.g., incubation for 2 h at room temperature in a PBS-containing solution such as PBS/Tween®). After incubation with the second labeled antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

In preferred embodiments, the second antibody may be labeled with one or more fluorescent or luminescent moieties, allowing detection of bound antigen by detection of an optical signal. Specific examples of fluorescent or luminescent labels are well known in the art and are described in more detail below.

In another exemplary embodiment, the preceding format may be altered by first binding the sample to the assay plate. Then, primary antibody is incubated with the assay plate, followed by detecting of bound primary antibody using a labeled second antibody with specificity for the primary antibody. Alternatively, the primary antibody may itself be labeled with a detectable tag.

Immunoassays for detecting prion protein may be either competitive or noncompetitive. Noncompetitive immunoassays are assays in which the amount of analyte is directly measured, such as the sandwich ELISA described above. In competitive assays, the amount of analyte present in the sample is measured indirectly by measuring the amount of an added (exogenous) analyte (PrP-Sc) displaced (or competed away) from a capture agent (anti PrP-Sc antibody) by the analyte present in the sample. In one type of competitive assay, a known amount of prion protein is added to the sample and the sample is then contacted with an antibody that specifically binds PrP-Sc. The amount of PrP-Sc bound to the antibody is inversely proportional to the concentration of prions present in the sample.

In preferred embodiment, the antibody is immobilized on a solid substrate. The amount of prion bound to the antibody may be determined either by measuring the amount of prion present in a prion/antibody complex, or alternatively by measuring the amount of remaining uncomplexed prion protein, for example by providing labeled prion protein or an analog thereof.

A hapten inhibition assay is another preferred competitive assay. In this assay prion protein is immobilized on a solid substrate. A known amount of anti-PrP-Sc antibody is added to the sample, and the sample is then contacted with the immobilized prion. In this case, the amount of anti-PrP-Sc antibody bound to the immobilized prions is inversely proportional to the amount of prions present in the sample. Again the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

In other embodiments, Western blot analysis may be used to detect and quantify the presence of PrP-Sc in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind PrP-Sc. The anti-prion antibodies specifically bind to PrP-Sc on the solid support. These antibodies may be directly labeled or alternatively may be subsequently detected using labeled antibodies (e.g., labeled sheep anti-mouse antibodies) that specifically bind to the anti-PrP-Sc.

Other assay formats include liposome immunoassays (LIA), which use liposomes designed to bind specific molecules (e.g., antibodies) and release encapsulated reagents or markers. The released chemicals are then detected according to standard techniques (Monroe et al., 1986). The skilled artisan will realize that the immunological methods of use in the practice of the present invention are not limited to those disclosed herein, but may include any immunodetection method known in the art.

Detergents and Other Means for Lipid Removal

Within the practice of the invention, a variety of detergents or other agents may be of use for removal of bound lipids from prion proteins and solubilization of delipidated prion protein. The skilled artisan will be familiar with denaturing and non-denaturing methods for removing bound lipids from proteins. Non-denaturing methods for lipid removal are generally preferred, as they will maintain the secondary and tertiary structure of PrP-Sc and PrP-C, reducing the cross-reactivity of various binding moieties for the two conformational forms of the protein.

Non-denaturing methods for removal of lipids from proteins are well known in the art. Use of a non-denaturing detergent, such as Triton X-100 and other Triton detergents, NP-40, Brij, Tween, octyl-β-thio-glucopyranoside, CHAPS, CHAPSQ, sodium cholate and other cholate type detergents and Genapol X-100, is a non-limiting example of such a method. Standard methods may be used to determine the optimal concentrations of detergents to use. In one such method, a concentration range of a particular detergent may be used to treat samples known to contain PrP-Sc. The effect of detergent concentration on the extent of bound lipid may be assayed by non-denaturing gel electrophoresis, followed by staining of the gel for the presence of phospholipid, sphingolipid or glycolipid bound to PrP-Sc. Alternatively, aqueous solutions containing micellar incorporated PrP-Sc may be treated with detergent and the amount of apparent PrP-Sc in solution that is available for antibody binding determined by standard immunoassay. It is a matter of routine experimentation for the skilled artisan to vary the length of exposure to detergent, detergent concentration, temperature, pH, salt concentration and other such factors to determine optimal conditions for detergent treatment of PrP-Sc. As an initial condition, the detergent treatment disclosed in Somerville and Carp (1983), incorporated herein by reference, may be of use for increasing immunodetection of PrP-Sc.

The skilled artisan will vary detergent concentration between 0.01 and 10.0%, more preferably 0.05 to 5%, more preferably 0.1 to 2.5%, more preferably 0.2 to 1.0%, more preferably 0.25 to 0.5% (vol/vol) to determine optimal detergent concentrations for use. As an initial starting condition, treatment at between 0° C. and 4° C., for between 5 min and 2 hr, pH 7.4 and isotonic salt concentration may be used. More preferably, length of treatment may vary between 10 min and 1.5 hr, more preferably between 15 min and 1.25 hr, more preferably between 30 min and 1 hr. Given the hydrophobic nature of PrP-Sc, it is possible that lowering salt concentration to between 10 to 50 mM or raising salt concentration to between 0.25 to 1.0 M may increase solubility of delipidated PrP-Sc. Addition of low concentrations of urea, DMSO or guanidinium isothiocyanate may also increase the solubility of PrP-Sc.

Non-limiting examples of detergents that could potentially be of use in the practice of the present invention, along with their critical micellar concentrations (CMC) are presented in Table 1 below. As an initial starting condition, use of a detergent at or close to its critical micellar concentration may be preferred for some applications.

TABLE 1
Detergents

| Detergent Name | CMC (mM) |
|---|---|
| APO-10 | 4.6 |
| APO-12 | 0.568 |
| BRIJ ™-35 (C₁₂E₂₃) | 0.09 |
| C₈E₆ | 9.9 |
| C₁₀E₆ | 0.9 |
| C₁₀E₈ | |
| C₁₂E₆ | 0.087 |
| C₁₂E₈ (Atlas G2127) | 0.11 |
| C₁₂E₉ | 0.08 |
| C₁₂E₁₀ (Brij 36T) | 0.2 |
| C₁₆E₁₂ | 0.0023 |
| C₁₆E₂₁ | 0.0039 |
| Cyclohexyl-n-ethyl-β-D-Maltoside | 120 |
| Cyclohexyl-n-hexyl-β-D-Maltoside | 0.56 |
| Cyclohexyl-n-methyl-β-D-Maltoside | 340 |
| n-Decanoylsucrose | 2.5 |
| n-Decyl-β-D-glucopyranoside | 2.2 |
| n-Decyl-β-D-maltopyranoside | 1.6 |
| n-Decyl-β-D-thiomaltoside | 0.9 |
| Digitonin | |
| n-Dodecanoyl sucrose | 0.3 |
| n-Dodecyl-β-D-glucopyranoside | 0.13 |
| n-Dodecyl-β-D-maltoside | 0.15 |
| Genapol C-100 | |
| Genapol X-80 | 0.06-0.15 |
| Genapol X-100 | 0.15 |
| HECAMEG | 19.5 |
| Heptane-1, 2, 3-triol | |
| n-Heptyl-β-D-glucopyranoside | 79 |
| n-Heptyl-μ-D-thioglucopyranoside | 30 |
| LUBROL PX^{TM} | 0.006 |
| MEGA-8 (Ocatanoyl-N-methylglucamide) | 58 |
| MEGA-9 (Nonanoyl-N-methylglucamide) | 19-25 |
| MEGA- 10 (Decanoyl-N-methylglucamide) | 6-7 |
| n-nonyl-β-D-glucopyranoside | 6.5 |
| Nonidet P-10 (NP-10) | |
| Nonidet P-40 (NP-40) | 0.05-0.3 |
| n-OctanoyL β-D-glucoslyamine (NOGA) | 80 |
| n-Octanoylsucrose | 24.4 |
| n-Octyl-alpha-D-glucopyranoside | 20 |
| n-Octyl-βD-glucopyranoside | 25 |
| n-Octy-β-D-maltopyranoside | 23.4 |
| PLURONIC F-68 | |
| PLURONIC F-127 | |
| THESIT | 0.1 |
| TRITON X-100 (tert-C₈-Ø-E_{9.6};like NP-40) | 0.3 |
| TRITON X-100 hydrogenated | 0.25 |
| TRITON X-114 (tert-C₈-Ø-E₇₋₈) | 0.35 |
| TWEEN ™20 (C₁₂-sorbitan-E₂₀;Polysorbate 20) | 0.059 |
| TWEEN ™40 (C₁₆-sorbitan-E₂₀) | 0.027 |
| TWEEN ™60 (C₁₈-sorbitan-E₂₀) | 0.025 |
| TWEEN ™80 (C_{18:1}-sorbitan-E₂₀) | 0.012 |
| n-Undecyl-β-D-maltoside | 0.59 |
| Caprylic acid, Na⁺salt (n-octanoate) | 351 |
| Cetylpyridinium chloride | 0.90 |
| CTAB (Cetyltri-methylammonium bromide) | 1.0 |
| Cholic acid, Na⁺salt | 4 |
| Decanesulfonic acid, Na⁺salt | 32.6 |
| Deoxycholic acid, Na⁺salt (DOC) | 1.5 |
| Digitonin | 0.087 |
| Dodecyltrimethyl-ammonium bromide | 14 |
| Glycocholic acid, Na⁺salt | 7.1 |
| Glycodeoxycholic acid, Na⁺salt | 2.1 |
| Lauroylsarcosine, Na⁺salt (Sarkosyl) | |
| Lithium n-dodecyl sulfate | 6-8 |
| Lysophosphatidyl-choline (16:0) | 0.007 |
| Sodium n-dodecyl sulfate (SDS, Lauryl sulfate, Na⁺salt) | 2.30 |
| Taurochenodeoxy-cholic acid, Na⁺salt | |
| Taurocholic acid, Na⁺salt | 3.3 |
| Taurodehydrocholic acid, Na⁺salt | |
| Taurodeoxycholic acid, Na⁺salt | 2.7 |
| Taurolithocholic acid, Na⁺salt | |
| Tauroursodeoxycholic Acid | |
| Tetradecyltrimethyl-ammonium bromide (TDTAB) | 3.5 |
| TOPPS | 4.5 |
| BigCHAP | 3.4 |
| CHAPS | 6-10 |
| CHAPSO | 8 |
| DDMAU | 0.13 |
| EMPIGEN ™BB (N-Dodecyl-N,N-dimethylglycine) | 1.6-2.1 |
| Lauryldimethylamine oxide | 1-3 |
| (LADAO, LDAO, Empigen OB) | |
| ZWITTERGENT ™3-08 | 330 |
| ZWITTERGENT ™3-10 | 25-40 |
| ZWITTERGENT ™3-12 (3-Dodecyl-dimethylammonio- | 2-4 |
| propane-1-sulfonate) | |
| ZWITTERGENT ™3-14 | 0.1-0.4 |
| ZWITTERGENT ™3-16 | 0.01-0.06 |

Non-detergent methods for removing bound lipid may include treatment with NDSB (Vuillard et al., 1998). Alternatively, alcohols, formaldehyde or other chemical treatments known in the art may be used. In other embodiments, lipid-coated PrP-Sc may be treated with lipase, phospholipase, sphingolipase or other enzymes known to degrade lipids in order to remove bound lipid from the protein.

The skilled artisan will be generally familiar with conditions for delipidation using organic solvents or enzymes. For organic solvents, using pure alcohol, formaldehyde or other solvents at room temperature or cooler, more preferably at 4° C. or cooler, more preferably at 0° C. or cooler, to obtain a miscible slurry of the aqueous and organic phase is preferred. The miscible slurry could be treated with lipases and the slurry directly analyzed to detect prions. Alternatively, the organic and aqueous phases could be separated by centrifugation or allowed to spontaneously separate. Depending on the solvent used, prions may be preferentially soluble in either the aqueous phase or the organic phase. Prions concentrated in an aqueous phase could be digested with lipases or other enzymes and then analyzed. Alternatively, enzymatic digestion could take place before exposure to organic solvent. As discussed above, removal of bound lipids from prions could result in a decrease in their aqueous solubility. Addition of low amounts of non-denaturing detergents or other solubilizing agents may be preferred to keep the delipidated prion proteins in solution. Although a wide range of concentrations and times of enzymatic digestion could be employed, treatment with 0.01 U/ml, 0.1 U/ml, 0.2 U/ml, 0.5 U/ml, 0.75 U/ml, 1.0 U/ml, 2.0 U/ml, or 5.0 U/ml for 5 min, 10 min, 20 min, 30 min, 45 min, 1 hr, 2 hr or 4 hr are preferred.

Labels

Certain embodiments of the present invention involve the attachment of label moieties to one or more molecules, e.g., to a primary or secondary antibody that binds to the analyte of interest (PrP-Sc). Alternatively, in some embodiments it may be desirable to label the analytes in a sample and to detect binding of labeled PrP-Sc to a binding moiety. Methods for attaching labels to antibodies or analyte proteins are well known in the art, as discussed in more detail below.

Non-limiting examples of fluorescent labels contemplated to be useful in practicing the present invention include: Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red. The skilled artisan will realize that the present invention is not limited to use of fluorescent labels, although in preferred embodiments binding of a labeled molecule is detectable by an optical signal. In other embodiments, the label may be radioactive (e.g., H³, C¹⁴, P³², I¹²⁵), calorimetric or enzymatic. Exemplary labels of use are known in the art.

Cross-Linking Reagents

In preferred embodiments, the binding moieties or analytes of interest may be attached to a surface by covalent or non-covalent interaction. In other preferred embodiments, labels may be attached to binding moieties or to analytes of interest, such as PrP-Sc. One means for promoting such attachments involves the use of chemical or photo-activated cross-linking reagents. Such reagents are known in the art and it is contemplated that any such reagent could be of use in the practice of the claimed invention.

Homobifunctional reagents that carry two identical functional groups are highly efficient in inducing cross-linking. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking molecules are disclosed in U.S. Pat. No. 5,603,872 and U.S. Pat. No. 5,401,511, incorporated herein by reference. Various ligands can be covalently bound to surfaces through the cross-linking of amine residues. Amine residues may be introduced onto a surface through the use of aminosilane, for example. Coating with aminosilane provides an active functional residue, a primary amine, on the surface for cross-linking purposes. In another exemplary embodiment, the surface may be coated with streptavidin or avidin with the subsequent attachment of a biotinylated molecule, such as an antibody or PrP-Sc. In preferred embodiments, ligands are bound covalently to discrete sites on the surfaces. To form covalent conjugates of ligands and surfaces, various cross-linking reagents have been used, including glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC).

In another non-limiting example, heterobifunctional cross-linking reagents and methods of using the cross-linking reagents are disclosed in U.S. Pat. No. 5,889,155. The cross-linking reagents combine, for example, a nucleophilic hydrazide residue with an electrophilic maleimide residue, allowing coupling in one example, of aldehydes to free thiols. The cross-linking reagent used can be designed to cross-link various functional groups.

Protein Chips

In preferred embodiments, the methods of the present invention may utilize arrays of binding moieties. Such arrays may be in the form of protein chips. Protein chip technology provides a means of rapidly screening analytes for their ability to bind to a potentially large number of binding moieties, such as antibodies, immobilized on a solid substrate. Specifically contemplated are array-based technologies such as those disclosed in U.S. Pat. Nos. 5,827,748; 6,192,168; 6,071,394; 5,858,804 and 5,948,627, each incorporated herein by reference. These techniques involve methods for analyzing large numbers of samples rapidly and accurately.

A protein chip consists of a solid substrate to which an array of binding moieties has been attached. For screening, the array is contacted with a sample containing analyte (for example PrP-Sc), which is allowed to bind. The degree of stringency of binding may be manipulated as desired by varying, for example, salt concentration, temperature, pH and detergent content of the medium. The chip is then scanned to determine which binding moieties have bound to the analyte. In certain embodiments, the structure of a protein chip may comprise: (1) an excitation source; (2) an array of binding moieties; (3) a sampling element; (4) a detector and (5) a signal amplification/data analysis system. A chip may also include a support for immobilizing the binding moieties.

In particular embodiments, an array of binding moieties may be tagged or labeled with a substance that emits a detectable signal. The tagged or labeled species may be fluorescent, phosphorescent, or luminescent, or it may absorb energy. When a binding moiety binds to a targeted analyte, a signal is generated that is detected by the chip. The signal may then be processed in several ways, depending on the nature of the signal. In certain embodiments, binding of analyte to binding moiety may be detected by quenching of a fluorescent, phosphorescent, or luminescent label attached to the binding moiety, for example by fluorescent resonance energy transfer (FRET). As discussed above, in alternative embodiments, the analyte itself may be labeled, or a second labeled binding moiety with affinity for the analyte may be added. In these embodiments, binding is detectable by the presence of an optical signal from the site of the bound analyte.

The binding moiety may be immobilized onto an integrated microchip that also supports a phototransducer and related detection circuitry. Alternatively, a binding moiety may be immobilized onto a membrane or filter that is then attached to the microchip or to the detector surface itself. The binding moieties may be directly or indirectly immobilized onto a transducer detection surface to ensure optimal contact and maximum detection. A variety of methods are known in the art to either permanently or removably attach binding moieties to a substrate. When immobilized onto a substrate, the binding moieties are stabilized and may be used repeatedly.

Exemplary substrates include nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane) and photopolymers which contain photoreactive species such as nitrenes, carbenes and ketyl radicals capable of forming covalent links with target molecules (e.g., U.S. Pat. Nos. 5,405,766 and 5,986,076).

Attachment of binding moieties to a selected support may be accomplished by any of several methods. For example, binding moieties may be bound to glass by first silanizing the glass surface, then activating with carbodiimide or glutaraldehyde. Alternative procedures may use reagents such as 3-glycidoxypropyltrimethoxysilane (GOP) or aminopropyltrimethoxysilane (APTS) linked via amino groups. With nitrocellulose membranes, the binding moieties may be spotted onto the membranes.

Specific binding moieties may first be immobilized onto a membrane and then attaching the membrane in contact with a transducer detection surface. This method avoids attaching the binding moieties to the transducer and may be desirable for large-scale production. Membranes that may be used include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BINDTM Costar, Cambridge, Mass.). In embodiments where the substrate must be optically transparent to allow for optical signal transmission through the substrate, glass or quartz are preferred.

Peptide Libraries

In certain embodiments, it may be desirable to identify random amino acid sequences in the form of a phage display library for use as binding moieties. Alternatively, it may be desirable to screen phage display libraries against PrP-C and PrP-Sc to identify peptide motifs that can preferentially bind to and/or stabilize specific conformations of PrP protein, or block the interaction between PrP-C and PrP-Sc. The phage display method has been used for a variety of purposes (e.g., Scott and Smith, 1990; DeGraaf et al., 1993; U.S. Pat. Nos. 5,565,332, 5,596,079, 6,031,071 and 6,068,829, each incorporated herein by reference).

Generally, a phage display library is prepared by first constructing a partially randomized library of cDNA sequences, encoding all possible amino acid combinations. The cDNA sequences are inserted in frame into, for example, a viral coat protein for a phage such as the fuse 5 vector (U.S. Pat. No. 6,068,829). The cDNAs are expressed as random amino acid sequences, incorporated into a coat protein such as the gene III protein of the fuse 5 vector. The randomized peptides are thus displayed on the external surface of the phage, where they can bind to analyte (PrP-C or PrP-Sc). Phage bound to the analyte may be separated from unbound phage using standard methods, for example by washing an array of analytes attached to a substrate. If desired, it is possible to collect bound phage, detach them from the analyte by exposure to an appropriate solution and proceed with another round of binding and separation. This iterative process results in the selection of phage with an increased specificity for the target analyte.

Once phage of an appropriate binding stringency have been obtained, it is possible to determine the amino acid sequence of the binding peptide by sequencing the portion of the phage genome containing the cDNA, for example by using PCR primers that flank the cDNA insertion site. Phage lacking any cDNA insert may be used as a control to ensure that binding is specific.

The skilled artisan will realize that phage display may be used to select for short (between 3 and 100, more preferably between 5 and 50, more preferably between 7 and 25 amino acid residues long) peptides that can bind to a desired analyte. Such peptides may be of use, for example, as potential inhibitors of PrP-Sc function.

Data Analysis

In certain embodiments, the present invention concerns use of data analysis for detection of prions and discrimination from other components of a potentially complex mixture obtained from a biological sample. In preferred embodiments, the data analysis methods are capable of distinguishing between PrP-C and PrP-Sc forms of the same protein. In embodiments where a single, monospecific antibody is used (e.g., an antibody that binds only to PrP-Sc and not to PrP-C or any other antigen), the data analysis consists simply of whether or not an antigen is present in the sample that binds to the antibody of interest. In more complex situations, multiple binding moieties, such as multiple monoclonal antibodies of different binding affinities and specificities may be used. In such cases, cross-reactivity against different antigens may be observed.

In preferred embodiments, it is anticipated that where an array of antibodies or other binding moieties is used, they will all have at least some affinity for PrP-C or PrP-Sc. Thus, cross-reactivity should be limited to proteins of similar amino acid sequence and/or secondary or tertiary structure. In a simplified model, certain antibodies in an array will bind only to PrP-C, certain antibodies will bind only to PrP-Sc, and certain antibodies will bind to either PrP-C or PrP-Sc. This situation might be complicated by the present of different strains or species-specific forms of PrP proteins (e.g., bovine and human PrP-Sc). In this simple format, the presence of PrP-Sc is indicated by a specific subset of antibodies that are known to bind to PrP-Sc standard proteins. The presence of PrP-Sc in a sample is indicated by antigen binding to that subset of antibodies. The array may be similarly calibrated against standard PrP-C and PrP-Sc proteins from different strains or species.

In the more complex situation, the presence of PrP-Sc in a sample does not result in a unique subset of antibodies binding to antigen. In this case, more complex methods of data analysis, such as by pattern recognition analysis, may be desired. The skilled artisan will realize that any form of data analysis that is capable of distinguishing between binding of PrP-Sc versus PrP-C or other antigens in the biological sample may be of use in the practice of the present invention. Non-limiting examples of pattern recognition methods are disclosed in U.S. Pat. Nos. 4,651,297; 6,117,193; 6,198,847 and 6,210,465, the relevant portions of each of which are incorporated herein by reference.

In general, an array of binding moieties (antibodies) may preferably be arranged as a two-dimensional matrix, wherein a specific antibody is located at a defined position on the array. This can be represented as a set of x,y coordinates, each of which corresponds to a single species of antibody. Where there are multiple copies of the single antibody present at each location, some may bind to antigen while others may not, depending on the affinity of the antibody for the antigen. Thus, in addition to the x,y coordinates for antibodies that bind to antigen in a sample, there may be an intensity value for each location, representing the number of individual antibody molecules bound to antigen and reflective of the affinity for antigen—the higher the affinity the more saturated with antigen the location will be. The data to be included in the pattern recognition analysis thus includes the location (x,y coordinate) of each antibody species that binds antigen, as well as the intensity of binding for each location. Including intensity information, it is possible to represent the data as a set of x,y,z values. The patterns of these values for the array may be determined in the presence of a variety of standard antigens, including PrP-C and PrP-Sc from different strains or species. The data may be stored in any convenient format, for example in a computer, for comparison to unknown samples. Analysis of the data may be performed by use of pattern recognition, neural network, or other analytical methods well known in the art.

Sample Collection

Various embodiments of the present invention involve analysis of samples for the presence of prions. In most embodiments, the samples are biological samples—either obtained directly from a human or non-human animal or suspected of containing material derived from a human or non-human animal. The skilled artisan will realize that the methods, compositions and apparatus of the present invention are of utility for screening a variety of different sample types for the presence of prions.

Within the scope of the present invention, many standard protocols are available for collection and preparation of biological samples. Such protocols may involve the collection or removal of intact cells and/or cell fragments by centrifugation or filtration, the removal of various contaminants by precipitation, extraction or enzymatic digestion and the separation of samples into fractions by various chromatographic procedures well known in the art. For the purposes of the present invention, the high-throughput analysis of samples is preferably obtained by keeping such sample treatment steps to a minimum.

In preferred embodiments, samples are collected directly from a human or non-human animal suspected of being infected with PrP-Sc. Although virtually any animal could be examined for the presence of PrP-Sc, in preferred embodiments the animals are those known to be carriers for TSEs, such as humans, cows, sheep, deer, elk or mink. Within the scope of the present invention, virtually any tissue, organ, cell type, fluid or other type of sample from a human or non-human animal could be analyzed. However, samples that may be obtained by minimally invasive techniques are preferred. Such samples could include blood, saliva, urine, semen, milk, lacrimal fluid, nasal secretions, lymphatic fluid, feces or even cerebrospinal fluid. Virtually any type of solid tissue sample may be analyzed, including without limitation adipose tissue (preferably obtained by a transcutaneous “punch”), lymph node, tonsil, tongue, esophagus, stomach, intestine, skin, muscle, heart, brain, peripheral nerve, pancreas, spleen, liver, lungs, kidneys, bladder, prostate, ovaries or any other solid tissue. Particularly preferred are tissues belonging to the reticuloendothelial system, such as the spleen.

In one non-limiting example, if prions are present in circulating leukocytes, a simple method to detect them would be to remove a blood sample from an individual, centrifuge it to collect cells, remove the “buffy coat” fraction of enriched white blood cells, lyse the cells and remove bound lipids from prion proteins as discussed above. An alternative embodiment would be to perform a transdermal punch to collect adipose tissue, lyse the cells and remove bound lipid from prion proteins. Other embodiments concern the use of hydrophobic-coated probes to collect prions from oral or rectal tissues.

Samples may be obtained before or after sacrifice of the subject animal. In the case of food animals such as cows or sheep, samples may be analyzed from slaughterhouse specimens before food processing. In other cases, such as asymptomatic humans or non-human animals, samples obtained from living subjects are preferred. Methods of non-lethal sample collection are well known in the art and any such method may be used in the practice of the invention. Preferred embodiments include needle biopsy samples, scrapings from the surface of various tissues or organs, punch samples, or samples obtained from various in vivo sampling techniques such as endoscopy, arterioscopy or laparoscopy.

Samples may be processed in various ways before analysis, including without limitation cooling, freezing, heating, homogenization, organic phase extraction, detergent extraction, enzymatic digestion, centrifugation, filtration, ultracentrifugation, ultrafiltration, lyophilization or various well known chromatographic procedures. In preferred embodiments, reverse phase chromatography or hydrophobic partitioning chromatography is used.

In particularly preferred embodiments, the hydrophobic properties of PrP-Sc are advantageously used to concentrate PrP-Sc for analysis. In a non-limiting example, a probe may be covalently or non-covalently coated with a hydrophobic substance that selectively adsorbs PrP-Sc. Covalent cross-linking of hydrophobic coatings onto probe surfaces may be performed as discussed above. The type of probe used is not important to the practice of the present invention—virtually any type of probe known in the art may be used to collect PrP-Sc. Non-limiting examples of probes that could be coated with hydrophobic substances include cotton-tipped swabs, wood probes, metal probes, plastic probes, glass probes or ceramic probes. Probes may be incorporated into complex apparatus for collection of samples from internal body cavities, organs or tissues.

The present invention is not limiting as to the type of hydrophobic substance that could be used as a coating. The only requirement is that it preferentially adsorb PrP-Sc. Preferred examples include plasminogen, apolipoprotein, detergent, lipid, alkanes, aromatic compounds, polycyclic aromatic compounds, long-chain alcohols and fatty acids.

In even more preferred embodiments, the hydrophobic coating may be attached to magnetic beads and the magnetic beads attached to the surface of a probe. After exposing the probe to a tissue surface, the hydrophobic substance attached to the beads may be collected by use of a magnet.

Although such hydrophobic coated probes may be used to collect prions from virtually any tissue surface or liquid, in preferred embodiments a hydrophobic probe may be used to swab the oral cavity, including tonsils, or rectal tissue. In other preferred embodiments, the hydrophobic substance may be attached to a chromatography support, over which a liquid sample is run. Virtually any type of liquid sample could be used, including blood. In this way, prions could be concentrated from very large volumes of liquid, essentially by performing a hydrophobic adsorption from liquid to a hydrophobic-coated solid surface. Using such methods it may be possible to detect prions at very low concentration in a liquid, such as blood. Using hydrophobic coated probes and swabbing oral or rectal tissues, it may also be possible to detect prions at low levels, prior to the development of overt disease symptoms.

Kits

All the essential materials and reagents required for the various aspects of the present invention may be assembled together in a kit. When the components of the kit are provided in one or more liquid solutions, the liquid solution preferably is an aqueous solution, with a sterile aqueous solution being particularly preferred.

Such kit components may comprise isolated primary and secondary antibodies with or without a label, reagents for developing and/or detecting a label, standard proteins such as PrP-C and/or PrP-Sc, buffers, detergents and any other compositions of use in the practice of the claimed invention. Such compositions may be liquid, frozen or lyophilized.

In other embodiments, kits could contain materials necessary for collection of samples. Such materials may include without limitation extraction solvents, swabs (including hydrophobic coated swabs), detergents and/or enzymes for delipidation of prions, and any other materials required to collect and process samples for detection of prions.

Where a portable biosensor is used, preferred kits would contain all materials needed for field use of the biosensor. Such materials would preferably be contained in color-coded containers, each color corresponding to a similarly colored loading port on the biosensor. In even more preferred embodiments, the materials could be preloaded into syringes or other devices for injection into loading ports. A matching color coding scheme would preferably be used with such injectors, corresponding to colored loading ports.

The components of the kit may be provided in dried or lyophilized forms. When reagents or components are provided as a dried form, reconstitution generally is by the addition of a suitable solvent, such as distilled water. It is envisioned that the solvent also may be provided in another container means.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the obtaining samples from one or more human or non-human animals. Such an instrument may be a syringe, pipette, forceps, scalpel, biopsy needle or virtually any type of probe. In preferred embodiments, the probes are hydrophobic coated probes, as described above. Additionally, instructions for use of the kit components is typically included.

Formulations and Routes for Administration to Patients

In certain embodiments, the peptides or other compositions of the present invention may be designed for administration to a subject. For example, a peptide could be designed to bind to and block the binding site(s) for interaction of PrP-C with PrP-Sc, preventing the conversion of additional molecules of PrP-C into prions and slowing or blocking disease progression. Alternatively, peptides could be designed to block potential receptor sites for uptake of PrP-Sc in the lining of the gastrointestinal tract. In principal, peptides could be designed that may bind to PrP-Sc and convert it back to the PrP-C conformation. Where such clinical applications are contemplated, it will be necessary to prepare pharmaceutical compositions—peptides, proteins, antibodies and/or drugs—in a form appropriate for the intended application. Generally, this will entail preparing compositions that are essentially free of pyrogens, as well as other impurities that could be harmful to humans or animals.

One generally will desire to employ appropriate salts and buffers to render delivery vectors stable and allow for uptake by target cells. Aqueous compositions of the present invention comprise an effective amount of proteins, peptides or antibodies, dissolved or dispersed in a pharmaceutically acceptable carrier or aqueous medium. Such compositions also are referred to as innocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions normally would be administered as pharmaceutically acceptable compositions.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Detection of Prions in Blood Samples

Blood samples are obtained and stored on ice or in a refrigerator at 4° C. prior to use. For extended storage, samples are flash frozen in liquid nitrogen and thawed immediately before analysis. Samples are centrifuged at 4° C. at 2,500 rpm in a refrigerated clinical centrifuge. The “buffy coat” layer containing enriched white blood cells is removed for analysis of prions.

The buffy coat is delipidated by forming a miscible slurry with chloroform, ethanol, isopropanol, hexafluoro-2-propanol or another organic solvent. The buffy coat is first diluted in phosphate buffered saline (PBS) containing 0.1% digitonin and lipase (0.25 U/ml). The miscible slurry is formed by vigorous shaking or vortexing with the organic solvent. After 30 min on ice, the slurry is analyzed for the presence of delipidated prion protein.

Prior to analysis, monoclonal antibodies against PrP-Sc, obtained from Prionics AF, Zurich, Switzerland, are covalently attached to the surface of glass slides as disclosed in U.S. patent application Ser. No. 09/974,089. Remaining non-specific binding sites on the glass surface are blocked by incubation with 0.1% bovine serum albumin (BSA) in PBS buffer. The slides containing bound anti-PrP-Sc antibody are then attached to a fluidics cell, as disclosed in U.S. patent application Ser. No. 09/974,089. Slurry containing delipidated prion protein is exposed to the antibody bound surface. The surface is washed twice with PBS. Prions attached to the anti-PrP-Sc antibody are detected by addition of a second anti-PrP-Sc antibody that is tagged with a fluorescent label, such as Texas Red, Rhodamine Red or any other fluorescent tag. The presence of bound PrP-Sc is detected by a CCD camera attached to the waveguide. The fluorescently tagged antibody is exposed to excitation light and the emission light is collected. Background fluorescence is determined by analysis of a control sample that is identically treated, but without any buffy coat sample. The background levels are subtracted from the experimental sample results to give corrected values for prion associated fluorescence.

### Example 2

### Use of Plasminogen

Samples are collected and treated as described in Example 1, except that the glass slide surface is covalently to plasminogen instead of to a first antibody. The delipidated prion sample is analyzed as in Example 1. The anti-PrP-Sc antibody obtained from Prionics AF is fluorescently labeled and used to detect PrP-Sc bound to plasminogen on the surface of the waveguide.

### Example 3

### Plasminogen Coated Swabs

Cotton-tipped swabs are coated with plasminogen covalently attached to magnetic beads and used to swab the tonsils of a subject. PrP-Sc adsorbed to the plasminogen coated beads is collected from with a magnet and delipidated as described in Example 1. PrP-Sc is detected as described in Example 1 or Example 2.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following literature citations as well as those cited above are incorporated in pertinent part by reference herein for the reasons cited in the above text.

Borman, Chem. & Eng. News, 76:22-30, 1998.

Campbell, In: Monoclonal Antibody Technology, Laboratory Techniques in Biochemistry and Molecular Biology, Burden and Von Knippenberg, Eds., Vol. 13:75-83, Elsevier, Amsterdam, 1984.

DeGraaf et al., Gene 128:13-17, 1993.

Fischer et al., Nature 408:479-483, 2000.

Gefter et al., Somatic Cell Genet., 3: 231-236, 1977.

Goding, In: Monoclonal Antibodies: Principles and Practice, 2d ed., Academic Press, Orlando, Fla., pp. 60-61, and 71-74, 1986.

Harlow and Lane, Antibodies: A Laboratory Manual, Cold Spring Harbor Laboratory, Cold Spring Harbor, N.Y., 1988.

Horwich and Weissman, Cell 89:499-510, 1997.

Kohler and Milstein, Nature, 256:495497, 1975.

Kohler and Mlstein, Eur. J. Immunol., 6: 511-519, 1976.

Prusiner, Proc. Natl. Acad. USA 95:13363-383, 1998.

Remington's Pharmaceutical Sciences, 15th ed., pp. 1035-1038 and 1570-1580.

Scott and Smith, Science 249:386-390, 1990

Somerville and Carp, J. Gen. Virol. 64:2045-50, 1983.

U.S. Pat. No. 4,196,265

U.S. Pat. No. 4,651,297

U.S. Pat. No. 5,401,511

U.S. Pat. No. 5,405,766

U.S. Pat. No. 5,565,332

U.S. Pat. No. 5,596,079

U.S. Pat. No. 5,603,872

U.S. Pat. No. 5,827,748

U.S. Pat. No. 5,858,804

U.S. Pat. No. 5,889,155

U.S. Pat. No. 5,948,627

U.S. Pat. No. 5,986,076

U.S. Pat. No. 5,998,149

U.S. Pat. No. 6,008,435

U.S. Pat. No. 6,031,071

U.S. Pat. No. 6,033,858

U.S. Pat. No. 6,068,829

U.S. Pat. No. 6,071,394

U.S. Pat. No. 6,117,193

U.S. Pat. No. 6,146,593

U.S. Pat. No. 6,165,784

U.S. Pat. No. 6,192,168

U.S. Pat. No. 6,198,847

U.S. Pat. No. 6,210,465

U.S. Pat. No. 6,214,565

What is claimed is:

1. A method of detecting prions in biological samples comprising:
a) obtaining a sample suspected of containing prions;
b) removing bound lipids from prion proteins; and
c) analyzing the sample for the presence of prion proteins.

2. The method of claim 1, wherein bound lipids are removed by treatment with a non-ionic detergent.

3. The method of claim 2, wherein the detergent is selected from the group consisting of Triton X-100, NP-40, Brij, Tween, octyl-glucopyranoside, octyl-β-thio-glucopyranoside, CHAPS, CHAPSQ, sodium cholate and Genapol X-80.

4. The method of claim 1, wherein bound lipids are removed by treatment with an alcohol.

5. The method of claim 1, wherein bound lipids are removed by treatment with a fixative.

6. The method of claim 5, wherein the fixative is formaldehyde.

7. The method of claim 1, wherein bound lipids are removed by treatment with an No n Detergent Sulfobetaines (NDSB).

8. The method of claim 1, wherein the sample is selected from the group consisting of blood, sputum, urine, milk, feces, lymphatic fluid, lymphatic tissue, adipose tissue and spleen tissue.

9. The method of claim 1, wherein the sample comprises cerebrospinal fluid.

10. The method of claim 1, wherein said analyzing comprises immunodetection.

11. The method of claim 10, wherein the immunodetection comprises ELISA or sandwich ELISA.

12. The method of claim 10, wherein the immunodetection comprises Western blotting, dot blotting or slot blotting.

13. A method of collecting prions for analysis comprising:
a) obtaining a probe coated with a hydrophobic substance; and
b) using the probe to collect prions.

14. The method of claim 13, further comprising analyzing the collected material for the presence of prions.

15. The method of claim 13, wherein the hydrophobic substance is selected from the group consisting of lipid, detergent, a hydrophobic synthetic polymer, plasminogen and apolipoprotein.

16. The method of claim 13, further comprising exposing the probe to oral or anal tissue to collect prions.

17. The method of claim 13, further comprising using the probe to swab the tonsils of a subject.

18. A method of detecting prions in biological samples comprising:
a) obtaining an array comprising at least two binding moieties, the binding moieties attached to discreet locations on the array;
b) exposing a biological sample suspected of containing a prion to the array; and
c) detecting binding to at least one binding moiety
wherein binding to the array is indicative of the presence of at least one prion in the sample.

19. The method of claim 18, wherein binding is detected to at least two binding moieties, further comprising identifying a pattern of binding to the array that is indicative of the presence of prions in the sample.

20. The method of claim 19, wherein the pattern of binding to the array distinguishes prions from non-infective proteins of identical amino acid sequence.

21. The method of claim 18, further comprising removing bound lipid from the prion protein.

22. The method of claim 18, wherein the binding moieties are antibodies.

23. The method of claim 18, wherein the binding moieties are peptides.

24. The method of claim 23, wherein the peptides are prepared from a phage display library.

25. A method of preparing antibodies against prions comprising:
a) obtaining a preparation of purified prions;
b) treating the prions to remove bound lipids; and
c) preparing antibodies against the prions.

26. An antibody prepared by the method of claim 25.

27. The method of claim 13, wherein the hydrophobic substance is attached to magnetic beads, the magnetic beads attached to the surface of the probe.

28. The method of claim 27, further comprising using a magnet to collect the beads from the probe after the prions have been collected.

29. The method of claim 28, wherein the hydrophobic substance is plasminogen.
